(19) United States
(12) Patent Application Publication
(10) Pub. No.: US 2010/0069918 A1
(43) Pub. Date: Mar. 18, 2010

(54) ORAL CLEANING DEVICE

(76) Inventor: Doanh NGUYEN, Cleveland, OH (US)

Correspondence Address: CURATOLO SIDOTI CO., LPA, 24500 CENTER RIDGE ROAD, SUITE 280, CLEVELAND, OH, 44145, US

(21) Appl. No.: 12/558,243

(22) Filed: Sep. 11, 2009

Related U.S. Application Data
(60) Provisional application No. 61/191,853, filed on Sep. 12, 2008.

(51) Int. Cl. A61B 17/22

(52) U.S. Cl. 606/127

(57) ABSTRACT

An oral cleaning device for removing tonsilliths lodged within the crypts of soft tissue within the buccal cavity which are typically the source of bad breath and the cause of sore throat is provided. The oral cleaning device includes a body, a head, and a contact member or portion for removing the tonsilliths through the application of pressure in a massage-like fashion. Operation of the oral cleaning device causes the crypts to contract in size which, in turn, causes the tonsilliths to be dislodged and expelled from the crypts.

### CROSS REFERENCE TO RELATED APPLICATIONS

This application claims the benefit of the filing date under 35 U.S.C. §119(e) from U.S. Provisional Application Ser. No. 61/191,853 filed Sep. 12, 2008, which is hereby incorporated by reference.

### TECHNICAL FIELD

Provided is an oral cleaning device and method for using an oral cleaning device, and particularly, to a device and method for removing debris from soft tissue within the buccal cavity.

### BACKGROUND

FIG. 1 shows a sagittal view of a human head 1 including the buccal cavity or mouth 2, palate 4, uvula 6, tongue 9 and tonsils 10. Many problems and diseases can arise within the soft tissue present within the mouth 2 and particularly within the tonsils 10. As shown in FIGS. 1 and 2, the tonsils 10 are small collections of lymphoid tissue that are generally almond shaped and are located at the opening of the oropharynx near the entrance to the throat 11. These tonsils 10 are known as the palatine tonsils 10. Within the nasopharynx are soft tissue structures known as adenoids or pharyngeal tonsils. The back of the tongue 9 contains soft tissue structures known as the lingual tonsils. These soft tissue structures within the pharynx form a ring which functions to trap bacteria and other pathogenic microorganisms preventing them from entering the body through the throat 11.

The tonsils 10 and other soft tissue structures of the mouth 2 do not possess an afferent lymphatic system. Instead, the tonsils 10 and other soft tissue structures of the mouth 2 contain many folds, crevices, and “pits” or cavities (hereinafter referred to as “the crypts 15”) which are arbor-like structures that allow the tonsils 10 and other soft tissue structures to capture and process antigens entering from the nasogastric tract. The crypts 15 are formed when squamous cell epithelium (which covers the tonsils 10) invaginates into the tonsils 10 (Passo, 2004). The interior epithelium of the crypts 15 is interspersed with lymphocytes, macrophages and dendritic cells which function to protect the body from foreign material (Passo, 2004). Typically, the average person has from about ten to about twenty crypts 15 within each of their two tonsils 10 (Passo, 2004).

Thus, the crypts 15 may contain caseous materials inside that have been known to contain leukocytes, epithelium cells, cellular debris, food debris, and colonizer bacteria, such caseous materials may include sulfur containing substrates that cause halitosis (bad breath) as they degrade. As shown in FIG. 3, this build-up of bacteria, particles, and other debris (hereinafter referred to as “the tonsillith 20” or “the tonsilliths 20”) generally appear as white or yellow spots on the tonsil 10 and may emit a foul odor causing bad breath. In addition, the presence of the tonsilliths 20 may lead to discomfort, sensation of a foreign body presence, and infection of the tonsils 10 (tonsillitis) which may lead to hyperemia, hypertrophy, and a sore throat 11.

There are many remedies available in the marketplace for addressing bad breath, such as mouthwashes, breath mints and other candies, breath-freshening sprays, and breath-freshening gums. While these remedies may mask the foul odor in the afflicted person's breath, the relief is often temporary and does not remove the tonsillith 20. It has previously been determined that tonsilliths 20 contain a variety of anaerobic bacteria, including Eubacterium, Fusobacterium, Megasphaera, Porphyromonas, Prevotella, Selenomonas and Tannerella (Tsuneishi, 2006). Prevotella and Fusobacterium have also been isolated from the tonsils of patients suffering from tonsillitis or sore throat 11 (Kuhn, 1995). Removal of the tonsilliths 20 may decrease the incident of sore throat 11. Techniques for removing the tonsilliths 20 include removing the tonsils 10 (tonsillectomy), opening up the crypts 15 through a tonsillotomy (i.e. laser cryptolysis), and application of a low frequency ultrasound. Attempts have been made to provide a device for the removal of tonsilliths 20 in a non-invasive manner, such as the device disclosed in U.S. Pat. No. 6,475,172 issued to Hall (hereinafter referred to as “the Hall device”). The Hall device requires a user to position the device adjacent to the tonsillith 20 and apply pressure to the surrounding tissue to remove the tonsillith 20. Other attempts have been made to remove tonsilliths 20, such as the device disclosed in U.S. Pat. Publication No. 2008/0155771 filed by Agbaje (hereinafter referred to as “the Agbaje device”). The Agbaje device is merely a brush that is swept over the tonsils 10 to remove any exposed tonsilliths 20. Therefore, the Hall and Agbaje devices are only capable of removing tonsilliths 20 that are on the surface of the tonsil 10 and visible to the user.

As shown in FIG. 3, however, many tonsilliths 20 are positioned within the crypts 15, and are not detectable upon a visual inspection of the tonsils 10. Therefore, the tonsilliths 20 may remain in the crypts 15 and continue to cause bad breath and infections without the person or user of the device knowing. In addition, application of too great a force with the Hall or Agbaje device to the tonsils 10 can damage the soft tissue of the tonsils 10. The Hall and Agbaje devices provide only a limited range of motion for removing the tonsilliths 20. Accordingly, there is a continuing need for a device with a wider range of motion to remove tonsilliths 20 located in difficult areas, and which can remove the tonsilliths 20 in a non-invasive manner, even when they are not detectable upon a visual inspection of the tonsils 10.

### BRIEF DESCRIPTION OF THE DRAWINGS

FIG. 1 is a sagittal view of a human head showing the location of the tonsils.

FIG. 2 is a front view of a mouth showing the orientation of the tonsils.

FIG. 3 is a cross sectional view of the tonsils showing exemplary crypts and tonsilliths;

FIG. 4A is a perspective view of a first exemplary embodiment of the oral cleaning device.

FIG. 4B is a side view of the exemplary embodiment of the oral cleaning device of FIG. 4A in a neutral position.

FIG. 4C is a side view of the exemplary embodiment of the oral cleaning device of FIG. 4A in a lower position.

FIG. 4D is a side view of the exemplary embodiment of the oral cleaning device of FIG. 4A in an upper position.

FIG. 5A is a side view of a second exemplary embodiment of the oral cleaning device extended to a first distance.

FIG. 5B is a side view of the exemplary embodiment of the oral cleaning device of FIG. 5A compressed to a second distance.

FIG. 6 is a side view of a third exemplary embodiment of the oral cleaning device.

FIG. 7A is a side view of a fourth exemplary embodiment of the oral cleaning device in a neutral position.

FIG. 7B is a side view of the exemplary embodiment of the oral cleaning device of FIG. 7A in a lower position.

FIG. 7C is a side view of the exemplary embodiment of the oral cleaning device of FIG. 7A in an upper position.

FIG. 8 is a perspective view of a filth exemplary embodiment of the oral cleaning device having a plurality of contact members and a vacuum suction.

FIG. 9 is a side view of a first exemplary embodiment of the contact members.

FIG. 10 is a front view of a second exemplary embodiment of the contact members.

FIG. 11 is a front view of a third exemplary embodiment of the contact members.

FIG. 12A is a side view of a sixth exemplary embodiment of the oral cleaning device and a cross sectional view of a tonsil with two tonsilliths.

FIG. 12B is a side view of the exemplary embodiment of the oral cleaning device of FIG. 12A as it is moved along the tonsil to dislodge the tonsilliths from the tonsil as depicted in FIG. 12A.

FIG. 13A is a side view of a seventh exemplary embodiment of the oral cleaning device in the lower position and a cross sectional view of a tonsil with two tonsilliths.

FIG. 13B is a side view of the exemplary embodiment of the oral cleaning device of FIG. 13A in a neutral position dislodging the tonsilliths from the tonsil as depicted in FIG. 13A.

FIG. 13C is a side view of the exemplary embodiment of the oral cleaning device of FIG. 13A in an upper position dislodging the tonsilliths from the tonsil as depicted in FIG. 13A.

FIG. 14A is a side view of an exemplary embodiment of the contact member of FIG. 9 and a cross-sectional view of a tonsil with two tonsilliths.

FIG. 14B is a side view of the exemplary embodiment of the contact member of FIG. 9 rotating to dislodge a tonsillith from the tonsil as depicted in FIG. 14A.

FIG. 14C is a side view of the exemplary embodiment of the contact member of FIG. 9 rotating to dislodge a tonsillith from the tonsil as depicted in FIG. 14A.

FIG. 15A is a side view of an eighth exemplary embodiment of the oral cleaning device in a lower position.

FIG. 15B is a side view of the exemplary embodiment of the oral cleaning device of FIG. 15A in an upper position.

FIG. 16A is a side view of a ninth exemplary embodiment of the oral cleaning device in a lower position.

FIG. 16B is a side view of the exemplary embodiment of the oral cleaning device of FIG. 16A in an upper position.

FIG. 17 is a sagittal view of a human head and a cross-sectional view of an exemplary embodiment of the oral cleaning device of FIG. 8 as applied to the tonsils.

FIG. 18 is a side view of a tenth exemplary embodiment of the oral cleaning device.

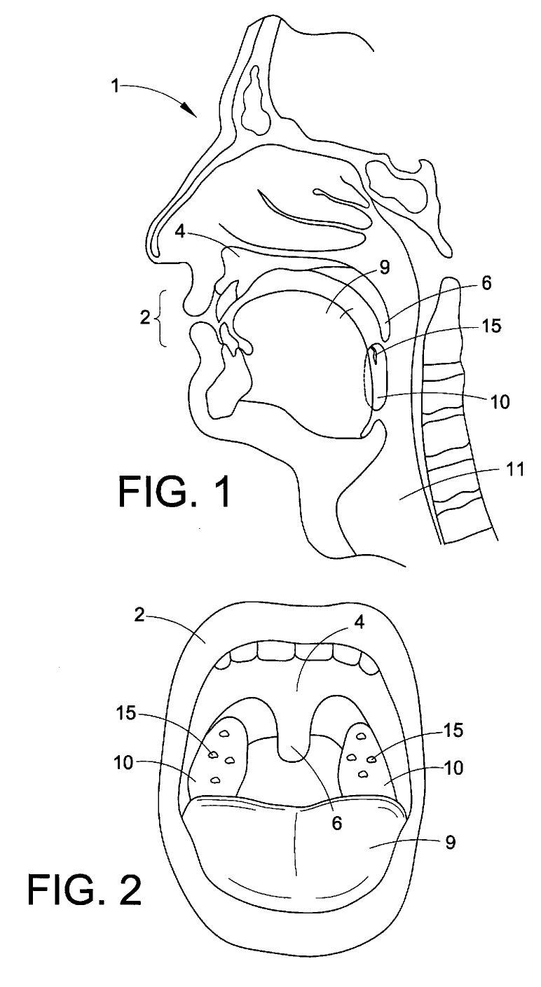

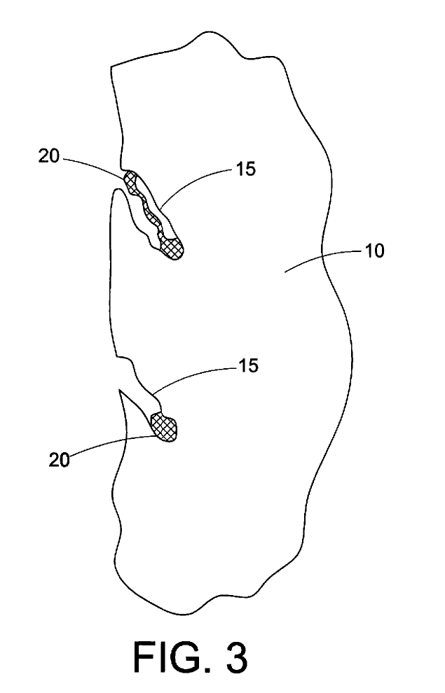

### DETAILED DESCRIPTION

Provided is an oral cleaning device for removing tonsilliths lodged in the crypts of soft tissue within the buccal cavity. The oral cleaning device comprises an elongated body having a first end, a second end, and a longitudinal axis to allow the oral cleaning device to reach the tonsilliths; and, a pivotable head attached to the first end of the body at a pivot point, said pivotable head having a contact member.

Also, provided is an oral cleaning device for removing tonsilliths lodged in the crypts of soft tissue within the buccal cavity. The oral cleaning device comprises an extendable elongated body having a first end, a second end, and a longitudinal axis to allow the oral cleaning device to reach the tonsilliths, the first end terminating in an integral head having a soft tissue contact portion.

Also, provided is an oral cleaning device for removing tonsilliths lodged in the crypts of soft tissue within the buccal cavity. The oral cleaning device comprises an elongated body having a first end, a second end, and a longitudinal axis to allow the oral cleaning device to reach the tonsilliths; and, a head attached to the first end of the body and carrying at least one contact member rotatable along the longitudinal axis of the elongated body.

Also provided is a process for removing tonsilliths lodged in the crypts in soft tissue within the buccal cavity comprising inserting an oral cleaning device into the buccal cavity, wherein the oral cleaning device comprises an elongated body having a first end and a second end, the body ergonomically designed to allow the oral cleaning device to reach the tonsilliths, and a head secured to the first end of the body, the head having at least one rotatable contact member; contacting the soft tissue with at least one contact member carried by the head of the oral cleaning device; rotating at least one contact member against the soft tissue to dislodge the tonsilliths present in the crypts of the soft tissue; and, removing the dislodged tonsilliths from the buccal cavity. According to certain exemplary processes, the process may further comprise biasing the at least one contact member against the soft tissue. According to certain exemplary processes, the process may further comprise moving the rotating contact member of the head along a soft tissue surface with an appropriate pressure in a massage-like fashion to remove the tonsilliths from the soft tissue.

Illustrative embodiments of the oral cleaning device and method will be described in further detail with reference to the drawing figures. It should be noted that the embodiments show in the drawing figures are intended to be merely illustrative and should not be considered to limit the oral cleaning device or method in any manner.

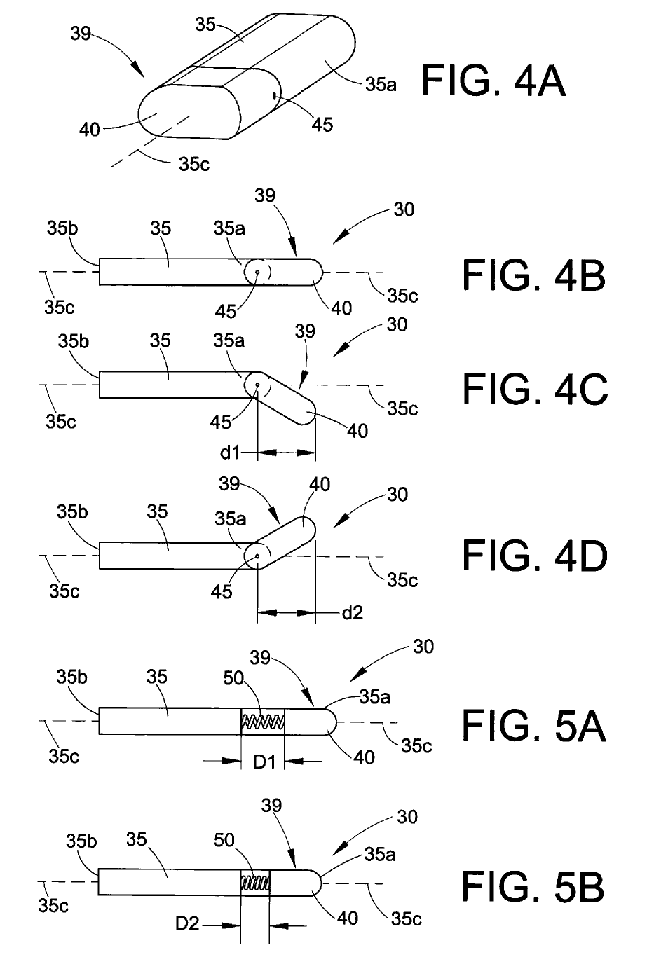

FIG. 4A shows a perspective view of one illustrative embodiment of an oral cleaning device 30 (hereinafter referred to as “the device 30”). As shown in FIGS. 4A-4D, the device 30 may be provided with an elongated body 35 and a head 39 having a contact member or contact portion 40 for removing the tonsilliths 20 from the tonsil 10.

Although shown as substantially rectangular, the elongated body 35 may be any shape and length allowing a user to contact the tonsils 10 with the contact member 40. Generally, the elongated body 35 has a first end 35a, a second end 35b, and a longitudinal axis 35c. The body 35 is ergonomically designed to allow the oral cleaning device 30 to reach the tonsilliths 20. In an illustrative example, the elongated body 35 may be curved to facilitate movement in and about the mouth 2 and tonsils 10. The elongated body 35 may be composed of any material, including, but not limited to, metals, plastics, composites, and the like. In one illustrative embodiment, the elongated body 35 is composed of a material, such as stainless steel, capable of undergoing sterilization processes. The elongated body 35 may further include any number of surface bumps, grips, grooves, handles, ridges, protrusions or the like which run either longitudinally along and/or circumferentially around the lateral section of the elongated body 35 to improve the handling of the oral cleaning device 30.

As shown in FIG. 4A, the head 39 is secured to the elongated body 35 so that the contact member 40 is capable of contacting the tonsils 10 to remove the tonsilliths 20 from the crypts 15. It is to be understood that the contact member 40 may be connected to or integral with the head 39. As shown in FIGS. 4B, 4C and 4D, the contact member 40 may be pivotally secured to the first end 35a of the elongated body 35 with a connection member such as a pin, hinge, or the like (hereinafter referred to as “the pin 45”) along a pivot. Such a configuration may facilitate a greater range of movement than possible with a fixed head 39, allowing for the head 39 and/or contact member 40 to be pivoted to a lower position relative to the longitudinal axis 35c of the elongated body 35, a neutral position relative to the longitudinal axis 35c of the elongated hod 35, and an upper position relative to the longitudinal axis 35c of the elongated body 35.

In an illustrative embodiment, the elongated body 35 may house a motor (not shown), such as an electric motor, capable of pivoting the head 39 and/or contact member 40, between a lower position (as shown in FIG. 4C) and an upper position (as shown in FIG. 4D). It is to be understood that the elongated body 35 may house a battery or include a plug to electronically connect the motor to an external source of power. However, it is to be further understood that a variety of different mechanisms may be provided to pivot the head 39 and/or contact member 40. For example, in an illustrative embodiment, the device 30 may be provided with a switch or trigger (not shown) that may be pulled or otherwise manipulated for pivoting the head 39 and/or contact member 40. In another illustrative embodiment, the head 39 and/or contact member 40 may be freely pivotable through angular manipulation and application of pressure when in contact with soft tissue within a subject's mouth 2. It is also to be understood that the head 39 may be capable of being rotated transverse to the longitudinal axis 35c of the elongated body 35 to provide increased maneuverability.

In an illustrative embodiment, the head 39 and/or contact member 40 may extend outward from the elongated body 35 a distance “d1” in the lower position (as shown in FIG. 4C) that is greater than the distance “d2” that the head 39 extends from the elongated body 35 in the upper position (as shown in FIG. 4D). Such a configuration may be provided to accommodate the curvature of the tonsils 10.

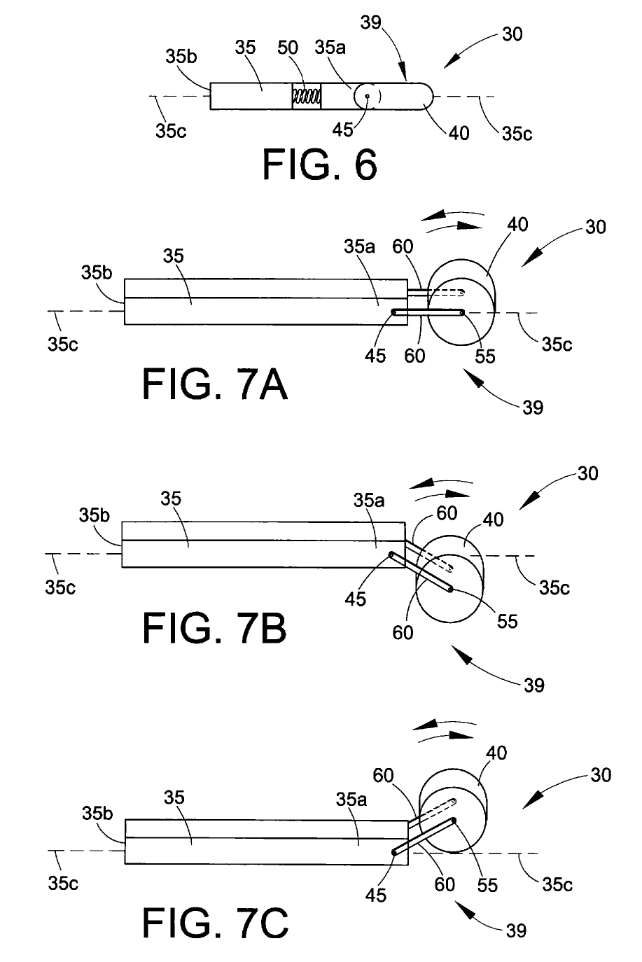

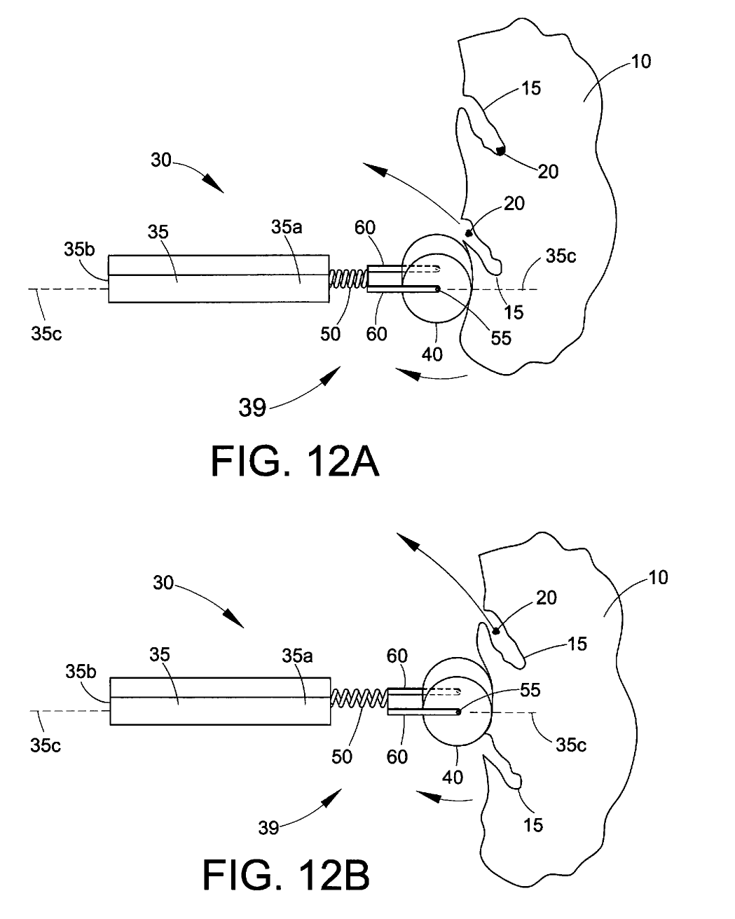

As shown in FIGS. 5A and 5B, the oral cleaning device 30 may simply comprise a biasing member or absorber 50 located within an elongated body 35 having an integral head 39 and contact member 40. The absorber 50 is capable of regulating the pressure applied to the tonsils 10 with the contact member 40. In an illustrative embodiment, the absorber 50 is a spring that may be compressed from a first distance “D₁” as shown in FIG. 5A, to a second distance “D₂” that is shorter than the first distance, to regulate the pressure applied to the tonsil 10. Although shown as integral with the elongated body 35, it is to be understood that the absorber 50 may be integral with the head 39, or positioned between the elongated body 35 and the head 39 (as shown in FIGS. 12A and 12B), and may be incorporated in any configuration of the oral cleaning device 30 herein described. In a non-limiting embodiment shown in FIG. 6, it is to be understood that the oral cleaning device 30 may include a head 39 that is pivotally secured to an elongated body 35 which includes an absorber 50.

According to certain embodiments, the contact member 40 is longitudinally rotatable relative to the longitudinal axis 35c of the elongated body 35 and is capable of removing tonsilliths when in contact with the soft tissue within the buccal cavity. The contact member 40 may be comprised of any material capable of contacting the tonsils 10 to remove the tonsilliths 20. Illustrative examples include metals, plastics, composites and the like. In an illustrative example, the contact member 40 is composed of a material, such as stainless steel, capable of undergoing sterilization processes. Although shown as substantially rectangular or oval-shaped, it is to be understood that the contact member 40 may be provided in a variety of shapes and sizes. Non-limiting examples of the contact member 40 include cylindrical, spherical, or barrel-shaped members as shown in FIGS. 7A, 7B and 7C. These contact members may have more than one different diameter. It is to be understood that the spherical or barrel-shaped contact members 40 may possess different diameters from a first end to a second end of the contact member 40. For example, contact member 40 may comprise a first end, a second end, and a middle portion between the first and second ends, wherein the middle portion has a diameter that is larger than the diameter of the first and second ends of the spherical or barrel-shaped contact head 40. It is also to be understood that the contact member 40 may be provided with one or more bumps, ribs, ridges, and the like, to massage the tonsil 10 and/or facilitate removal of the tonsilliths 20.

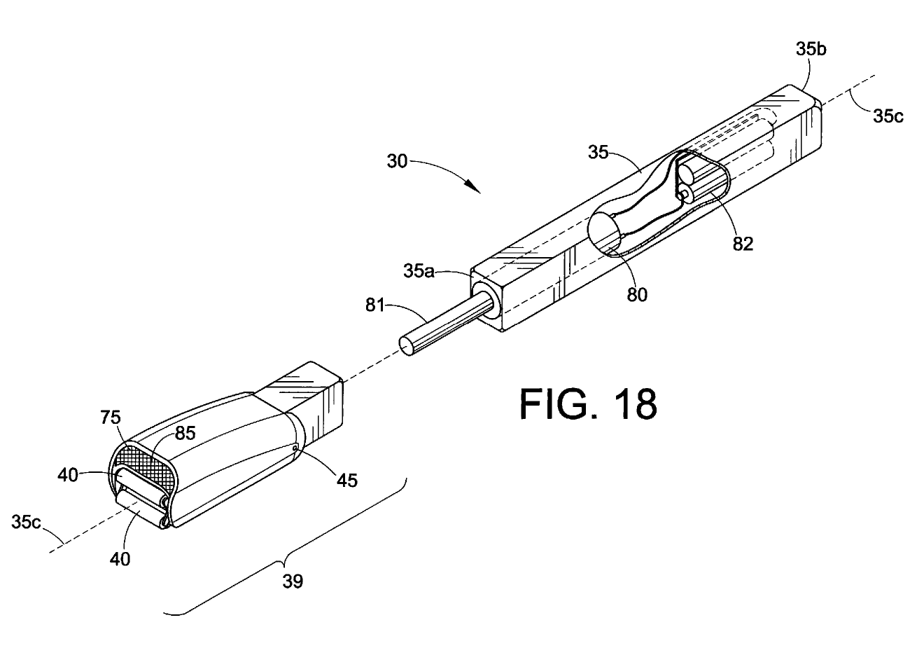

In another illustrative embodiment, the head 39 and the contact member 40 may be removeably secured to the elongated body 35 and interchangeable with a variety of different shapes and sizes of heads 39 and contact members 40. In yet another illustrative embodiment, the head 39 and/or contact member 40 may be interchangeable with other fittings including, but not limited to, toothbrushes, dental floss, and the like. In a non-limiting embodiment, the device 30 may be provided with a motor 80 (as shown in FIG. 18) that is capable of operating each respective fitting when connected to the elongated body 35. For example, the motor 80 may be capable of operating a toothbrush fitting as an electric toothbrush.

In one illustrative embodiment, the head 39 comprises a space between longitudinally divided first and second arms. Each of the first and second arms have a first end and a second end. Contact member 40 is attached to the first ends of the arms and is positioned within the space between the first and second arms. Thus, the contact member 40 is positioned between the two spaced apart arms. At least one contact member 40 is attached to the first ends of the first and second arms of the head 39 along an axis and is freely rotatable about the axis in either or both a forward or reverse direction along the longitudinal axis of the elongated body 35. In one illustrative embodiment, at least two contact members 40 are rotatably mounted between the first ends of the first and second arms of the head 39 along an axis. As shown in FIG. 7A, the head 39 may include one or more arms 60 securing the contact member 40 to the elongated body 35. The arm 60 may be secured to a rotatable contact member 40 to allow the contact member 40 to roll across the tonsil 10, thereby reducing friction and risk of injury or damage to the soft tissue. For example, the contact member 40 may be rotatably connected to the arms 60 with a pin 55, bearing, or the like. Although shown as connected to the elongated body 35 with one or more arms 60, it is to be understood that the contact member 40 may be rotatably secured to the elongated body 35 in a variety ways. As shown in FIGS. 7B and 7C, such a configuration may also provide that the arms 60 are pivotable about the pin 45, and/or include at least one type of absorber 50 as depicted in FIGS. 5A, 5B, 6, 12A and 12B.

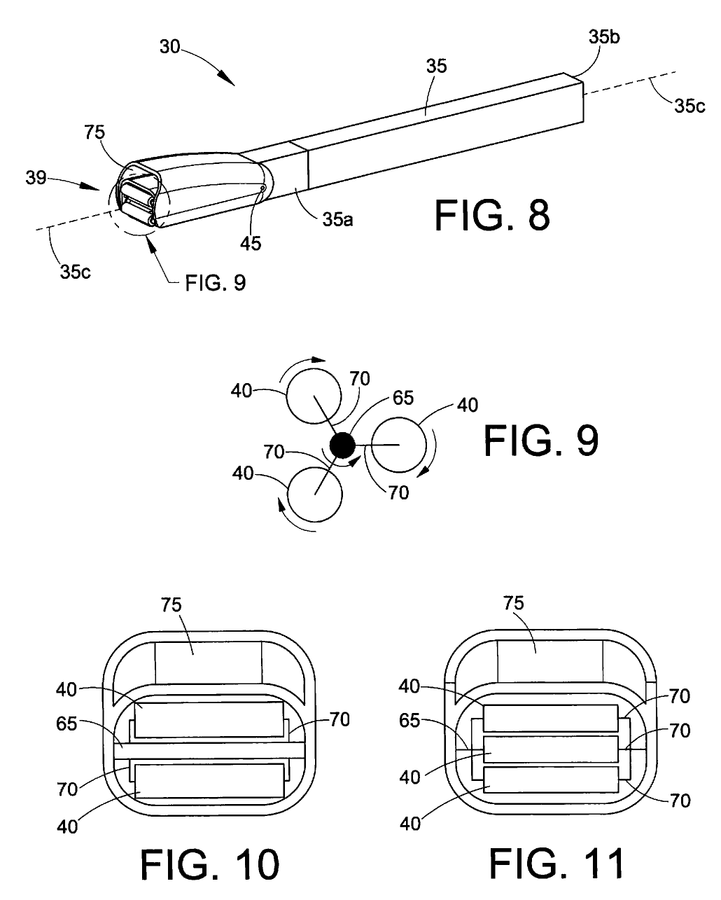

In another illustrative embodiment, as shown in FIGS. 8-11, the head 39 may include a plurality of contact members 40. Although shown as having three contact members 40, it is to be understood that any number (a plurality) of contact members 40 may be provided. The plurality of contact members 40 may be rotatably arranged about an assembly axis, wherein rotation of the assembly of the contact members 40 is driven by a switch, trigger, motor, or the like (not shown) allowing free rotation of each contact member 40 against the soft tissue about the contact member axis. As shown in FIGS. 9, 10, and 11 the contact members 40 may be secured to a drive member 65 via arms 70 in an arm and pin assembly. The drive member 65 may be operated by a switch, trigger, motor or the like (not shown). It is to be understood that as the drive member 65 rotates, the arms 70 rotate, causing the contact members 40 to be rotated about the arms 70. It is to be further understood that the contact members 40 may be rotatably secured to the arms 70 so that the contact members 40 may roll or otherwise freely rotate across the tonsils 10 to minimize frictional forces against the tonsillar tissue which could result in abrading the tissue or other tissue damage. FIGS. 10 and 11 provide non-limiting examples of different configurations for the drive member 65, the arms 70, and the contact member 40 in an arm and pin assembly. Specifically, FIG. 10 shows an illustration of the arm and pin assembly of the contact member 40 wherein the drive member 65 comprising a rotating shaft while FIG. 11 shows an illustration of the arm and pin assembly of the contact member 40 wherein the drive member 65 comprises a hub and spoke assembly directly connected to the arms 70 and contact member 40.

It is to be understood that a variety of other features may be provided with the oral cleaning device 30. In an illustrative embodiment, as shown in FIG. 8, the oral cleaning device 30 may be provided with at least one vacuum suction port 75 in fluid communication with a vacuum (not shown). The vacuum suction port 75 may be located proximate to the contact member 40 for removing tonsilliths 20 dislodged from the crypts 15 of the soft tissue. Accordingly, after the tonsilliths 20 are removed from the tonsils 10 with the contact member 40, the tonsilliths 20 may then be removed from the mouth 2 via the vacuum suction port 75. In another illustrative embodiment, the oral cleaning device 30 may be provided with a fluid dispenser (not shown) in fluid communication with a liquid source, such as water, to rinse off the tonsilliths 20 and/or otherwise cleanse the treated area. In yet another illustrative embodiment, an ultrasonic device (not shown) may be provided to facilitate removal of the tonsilliths 20 from the crypts 15. It is to be understood that such features may be used alone with the oral cleaning device 30, or in any combination.

Turning to the oral cleaning device 30, an illustrative method of using the oral cleaning device 30 is depicted in FIGS. 12A through 14C set forth below. As shown in FIGS. 12A and 12B, the contact member 40 may be positioned adjacent the lower portion of the tonsil 10 near the throat 11. The contact member 40 may be pressed against the tonsil 10 and raised upward toward the palate 4 such that the contact member 40 applies pressure to the crypts 15 to remove the tonsilliths 20 therefrom (for example, by contracting the crypts 15 to dislodge and expel the tonsillith 20). The contact member 40 may be freely rotatable about the pin 55 to minimize friction with the tonsil 10. A biasing member or absorber 50 is located between the first end 35a of the body 35 and the head 39 to regulate the pressure or force applied to the tonsils 10. It is to be understood that the process may be repeated and that the contact member 40 may be moved up and down, or side to side, along the tonsils 10 in a massaging fashion.

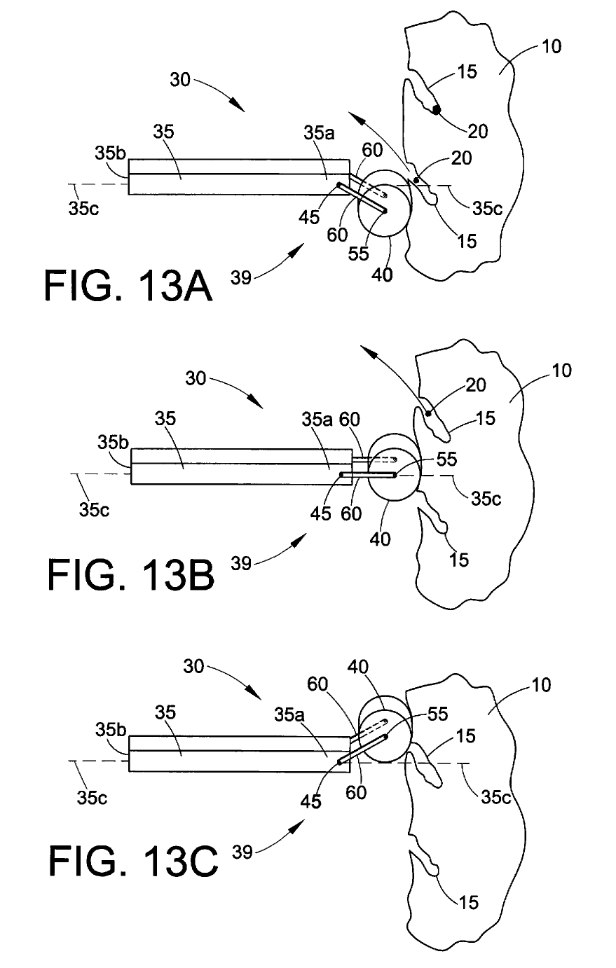

In another illustrative embodiment, as shown in FIGS. 13A, 13B and 13C, the head 39 may be pivotably connected to the elongated body 35 to provide additional mobility. As shown in FIG. 13A, the contact member 40 may be positioned adjacent the lower portion of the tonsil 10 near the throat 11. The contact member 40 may be pressed against the tonsil 10 and raised upward along the tonsil 10 toward the palate 4 such that the contact member 40 applies pressure to the crypts 15 to remove the tonsilliths 20 therefrom (see FIGS. 13B and 13C). The contact member 40 may be rotatable about the pin 55 to minimize friction with the tonsil 10. It is to be understood that the process may be repeated and that the contact member 40 may be moved up and down, or side to side, along the tonsils 10 in a massaging fashion. According to the embodiment shown in FIGS. 13A-13C, the contact member 40 is connected to the body 35 at pivot point 45 via spaced apart arms 60. During use of the device, the arms 60 and contact member 40 pivot at point 45 from an upper, neutral, and lower positions.

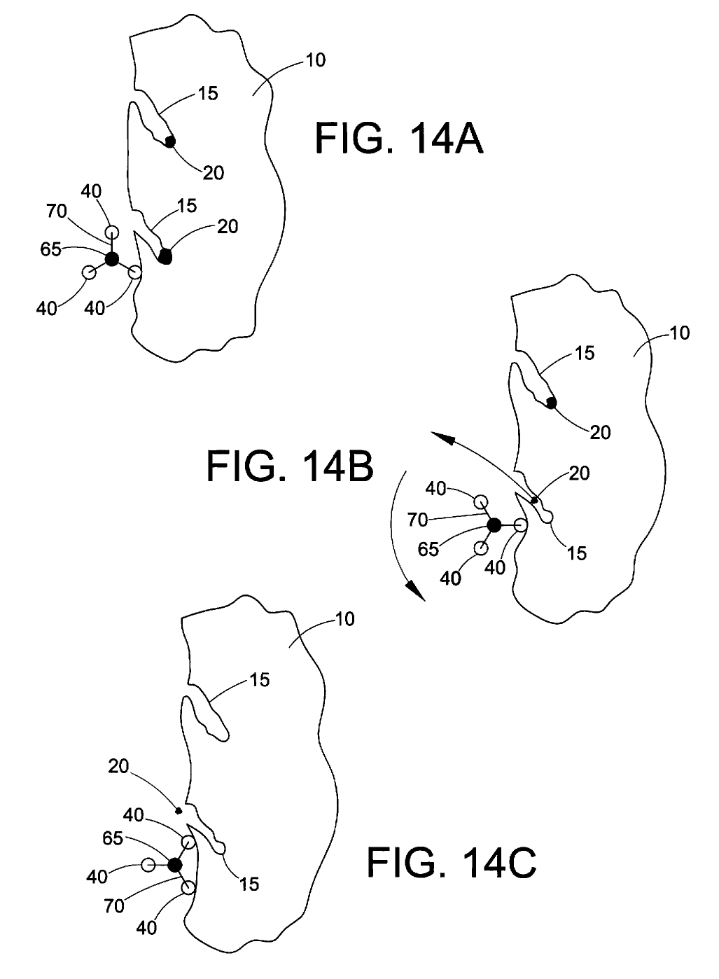

In another illustrative embodiment, as shown in FIGS. 14A, 14B and 14C, the head 39 may be provided with an assembly comprising multiple contact members 40. As shown in FIG. 14A, the contact members 40 may be positioned adjacent the lower portion of the tonsil 10 near the throat 11. The contact members 40 may be pressed or contacted against the tonsil 10 and the drive member 65 activated to rotate the arms 70 and contact members 40. The contact members 40 are freely rotatable about the arms 70 to minimize friction with the tonsil 10. As shown in FIGS. 14A, 14B and 14C, the contact members 40 apply pressure to the crypts 15 to dislodge the tonsilliths 20 therefrom. It is to be understood that the head 39 may be raised (or pivoted) upward along the tonsil 10 toward the palate 4 as described above. It is to be understood that the process may be repeated and that the contact member 40 may be moved up and down, or side to side, along the tonsils 10 and that such movement may be accomplished through the free rotation of the contact member 40. The user of the oral cleaning device may bias the at least one contact member against the soft tissue with an appropriate pressure to remove the tonsilliths 20 with minimal irritation and damage to the soft tissue. The biasing may be accomplished in a massage-like fashion to remove the tonsilliths 20 from the soft tissue. Finally, the tonsilliths 20 are removed from the buccal cavity or mouth 2 either manually or through a vacuum suction port 75 as depicted in FIG. 8.

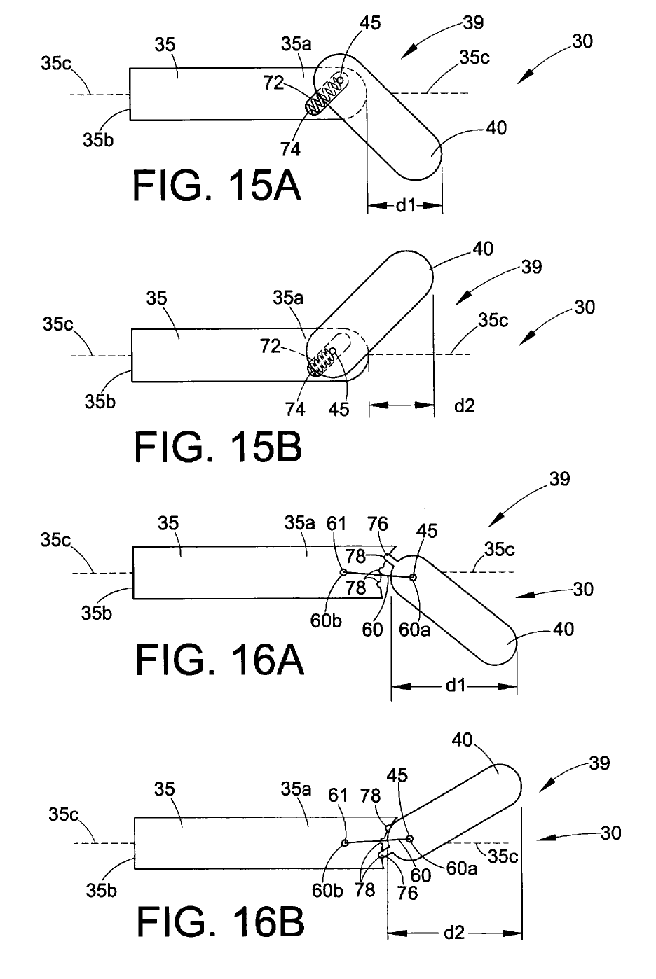

In another illustrative embodiment, as shown in FIGS. 15A and 15B, the first end 35a of the elongated body 35 may be provided with at least one groove, opening or slot 72 for moveably securing the head 39 to the elongated body 35. Such a configuration allows for movement of the head 39 in relation to the elongated body 35 of the oral cleaning device 30. As shown in FIG. 15A, pin 45 may extend through the slot 72 to secure the head 39 to the elongated body 35. A biasing member 74, such as a spring, may be provided to position the head 39 along the slot 72. As the head 39 is pivoted about the pin 45, as described above, the biasing member 72 may position the head 39 (and/or contact member 40) along the slot 72 so that the head 39 extends outward from the elongated body 35 a distance “d1” in the lower position (as shown in FIG. 15A) that is greater than the distance “d2” that the head 39 (and/or contact member 40) extends from the elongated body 35 in the upper position (as shown in FIG. 15B). It is to be understood, however, that one of ordinary skill in the art will appreciate that a variety of different configurations of the biasing member 74 and/or slot 72 may be provided.

In yet another illustrative embodiment, head 39 of the oral cleaning device 30 may be secured to the body with at least one connection member allowing for the head to be pivoted in relation to the body. As shown in FIGS. 16A and 16B, the connection member may secure the head 39 to the elongated body 35 with one or more arms 60 in at least one arm and pin assembly. The arms 60 generally have a first end 60a and a second end 60b, the first end 60a of the arm 60 attached to the head 39 with a first pin 45 and the second end 60b of the arm 60 attached to the elongated body 35 with a second pin 61. The arm and pin assembly allows for the head 39 to be pivoted in relation to the elongated body 35. The head 39 may be provided with a protrusion 76 and the first end 35a of the elongated body 35 may be provided with one or more recesses 78 capable of receiving the protrusion 76. The protrusion 76 and recess 78 when engaged prevent the arm and pin assembly from pivoting the head 39 in relation to the body 35. The arm 60 may be raised (for example, with a switch, trigger, motor, or the like) about pivot point 45 and/or pivot point 61 so that the head 39 (and/or contact member 40) may extend outward from the body 35 a distance “d1” in the lower position (as shown in FIG. 16A) that is greater than the distance “d2” that the head 39 (and/or contact member 40) extends from the elongated body 35 in the upper position (as shown in FIG. 16B).

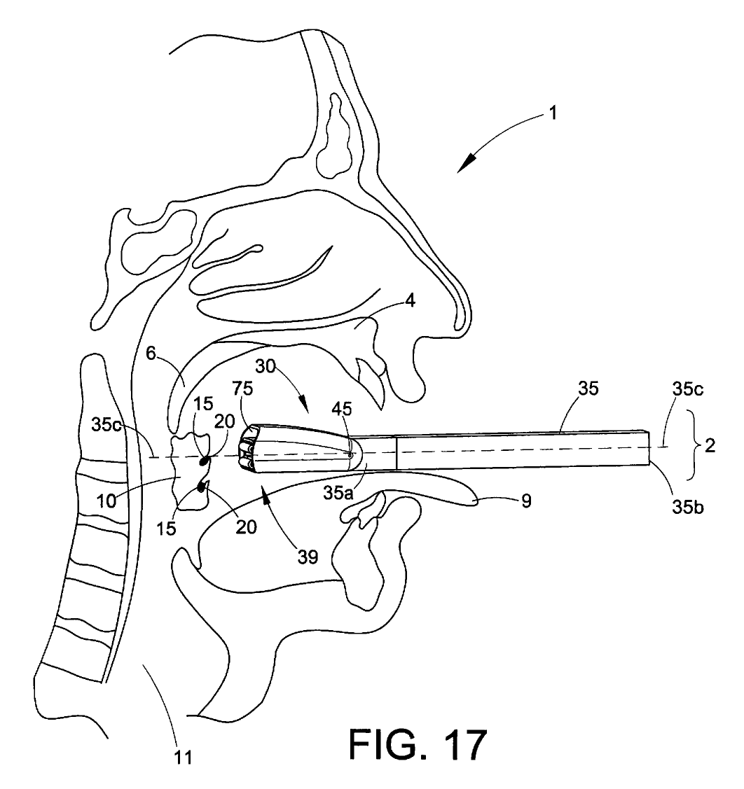

FIG. 17 shows the oral cleaning device 30 inserted into a subject's buccal cavity or mouth 2. The subject's tongue 9 may be sticking out of the subject's mouth 2 and the subject's head 1 may be tilted back to allow for easier access to remove the tonsilliths 20 from the soft tissue. It is to be understood that the head 39 of the oral cleaning device 30 may be applied to a lower end of the tonsil 10 and moved upward along the tonsil 10 toward the palate 4 as described above. Also, it is to be understood that the head 39 of the oral cleaning device 30 may be pivoted to a lower position, a neutral position, or an upper position along pivot point 45. It is to be understood that this process may be repeated and that the contact member 40 may be moved up and down, or side to side, along the tonsils 10.

As shown in FIG. 18, the oral cleaning device 30 may include a suction chamber 85 for collecting the tonsilliths 20 that are suctioned into the oral cleaning device 30 from the vacuum port 75. The vacuum suction chamber 85 and/or head 39 may be detachable from the oral cleaning device 30 allowing for cleaning of the tonsilliths 20 from the vacuum suction chamber 85. As shown in FIG. 18, the oral cleaning device 30 may contain an electric motor 80 powered by a battery 82 and rotatable shaft 81 for longitudinally rotating the contact member 40 relative to the longitudinal axis 35c of the elongated body 35. The electric motor 80 and/or head 39 may include a plurality of gears (not shown) as required to longitudinally rotate the contact member 40 relative to the longitudinal axis 35c of the elongated body 35.

The oral cleaning device 30 as described above may be used directly by the person whose mouth is to be treated or by a third party. Examples of third party's who may use the oral cleaning device 30 include health care professionals such as dentists or physicians and their assistants as well as laymen. The oral cleaning device 30 may be used on a regular basis as desired such as every day, once a week, etc., or as required to treat halitosis and/or sore throat 11. The person treated with the oral cleaning device 30 may rinse his or her mouth after each use to further remove any dislodged tonsilliths. The oral cleaning device 30 may be washed with soap and water or sterilized with any suitable cleanser after each use as desired. The oral cleaning device 30 may be fitted with a detachable head for use on multiple subjects.

While the oral cleaning device 30 have been described above in connection with certain illustrative embodiments, it is to be understood that other similar embodiments may be used or modifications and additions may be made to the described embodiments for performing the same function without deviating therefrom. Further, all embodiments disclosed are not necessarily in the alternative, as various embodiments may be combined or subtracted to provide the desired characteristics. Variations can be made by one having ordinary skill in the art without departing from the spirit and scope hereof. Therefore, the oral cleaning device 30 should not be limited to any single embodiment, but rather construed in breadth and scope in accordance with the recitations of the attached claims.

I claim:

1. An oral cleaning device for removing tonsilliths lodged in crypts of soft tissue within a buccal cavity comprising:
an elongated body having a first end, a second end, and a longitudinal axis to allow the oral cleaning device to reach the tonsilliths; and,
a pivotable head attached to the first end of the body at a pivot point, said pivotable head having a contact member.

2. The oral cleaning device of claim 1, wherein the pivotable contact member is attached to the body by a connection member positioned in an opening formed in the body.

3. The oral cleaning device of claim 2, wherein a biasing member is located within the opening and is capable of positioning the head along the opening.

4. The oral cleaning device of claim 3, wherein the biasing member comprises a spring.

5. The oral cleaning device of claim 4, wherein a second biasing member is located within the elongated body and is capable of extending and contracting the length of the elongated body to regulate the pressure applied to the soft tissue.

6. The oral cleaning device of claim 1, wherein the head is pivotable to at least an upper position and a lower position, the head pivotable outwardly from the body at a distance in the lower position that is greater than the distance that the head extends from the body in the upper position.

7. The oral cleaning device of claim 1, wherein the pivotable head includes a protrusion and the first end of the body includes at least one recess capable of receiving the protrusion, wherein engaging the protrusion in the recess prevents the connection member from pivoting the head in relation to the body.

8. The oral cleaning device of claim 4, further comprising a switch, trigger, or motor capable of biasing the spring.

9. The oral cleaning device of claim 1, further comprising a switch, trigger, or motor capable of pivoting the contact member.

10. The oral cleaning device of claim 1, wherein the elongated body comprises at least one bend point.

11. An oral cleaning device for removing tonsilliths lodged in crypts of soft tissue within a buccal cavity comprising:
an extendable elongated body having a first end, a second end, and a longitudinal axis to allow the oral cleaning device to reach the tonsilliths, the first end terminating in an integral head having a soft tissue contact portion.

12. The oral cleaning device of claim 11, comprising a biasing member located within the elongated body which is capable of extending and contracting the length of the elongated body to regulate the pressure applied to the soft tissue.

13. The oral cleaning device of claim 12, wherein the biasing member comprises a spring.

14. An oral cleaning device for removing tonsilliths lodged in crypts of soft tissue within a buccal cavity comprising:
an elongated body having a first end, a second end, and a longitudinal axis to allow the oral cleaning device to reach the tonsilliths; and,
a head attached to the first end of the body and carrying at least one contact member rotatable in the longitudinal axis of the elongated body.

15. The oral cleaning device of claim 14, wherein the head is attached to the first end of the body of the oral cleaning device at a pivot point.

16. The oral cleaning device of claim 15, wherein the head comprises spaced apart first and second arm, each of the arms having a first end and a second end, wherein the at least one contact member is attached to the first ends of the arms and is positioned within the space between the arms.

17. The oral cleaning device of claim 16, wherein the at least one contact member is attached to the first ends of the first and second arms along an axis and is freely rotatable about the axis in either or both of a forward and reverse direction along the longitudinal axis of the elongated body.

18. The oral cleaning device of claim 15, wherein the head is attached to the body by a connection member located in an opening formed in the body.

19. The oral cleaning device of claim 17, wherein a biasing member is located within the opening and is capable of positioning the head along the opening.

20. The oral cleaning device of claim 17, wherein at least two contact members are rotatably mounted between the first ends of the first and second arms of the head along an axis.

21. The oral cleaning device of claim 20, wherein the contact members are cylindrical, spherical or barrel-shaped.

22. The oral cleaning device of claim 15, wherein a biasing member is located within the elongated body and is capable of extending or contracting the length of the elongated body to regulate the pressure applied to the soft tissue.

23. The oral cleaning device of claim 15, wherein the contact member comprises
a rotatable drive shaft;
a plurality of arms connected to the rotatable drive shaft; and
freely rotatable contact members engaged with the arms.

24. The oral cleaning device of claim 23, wherein the rotatable drive shaft is driven by a motor.

25. The oral cleaning device of claim 21, wherein the rotatable contact member comprises more than one different diameter.

26. The oral cleaning device of claim 23, further comprising at least one biasing member for regulating the pressure applied to the soft tissue by the contact member.

27. The oral cleaning device of claim 14, wherein the head is pivotable to at least an upper position and a lower position, the head pivotable outwardly from the body at a distance in the lower position that is greater than the distance that the head extends from the body in the upper position.

28. The oral cleaning device of claim 14, further comprising at least one vacuum port located proximate to the contact member for removing tonsilliths dislodged from the crypts of the soft tissue.
